(19) United States
(12) Patent Application Publication
(10) Pub. No.: US 2009/0030492 A1
(43) Pub. Date: Jan. 29, 2009

(54) NERVE LEAD TIE DOWN WITH BEARING

(75) Inventors: Donnie Welty, Danbury, TX (US); Bryan Byerman, League City, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC., Houston, TX (US)

(21) Appl. No.: 11/829,595

(22) Filed: Jul. 27, 2007

(51) Int. Cl. A61N 1/04

(52) U.S. Cl. 607/115

(57) ABSTRACT

A lead tie-down for limiting movement of an electrical lead from an implantable medical device is disclosed. Some system embodiments include a lead assembly having an electrical lead and a stimulating electrode and a lead tie-down coupled to the electrical lead and configured for coupling to surrounding tissue. The lead tie-down permits rotational movement of the electrical lead relative to the lead tie-down. In some embodiments, the lead tie-down includes a clamping member having a bore therethrough. The bore is configured to receive an electrical lead extending from an implantable medical device and to permit rotation of the lead within the clamping member.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

1. Field of Art

The subject matter of this disclosure relates generally to the field of medical devices. More particularly, the present disclosure relates to implantable medical devices. Even more particularly, the present disclosure relates to leads used in connection with implanted neurostimulation devices.

2. Description of Related Art

Various diseases and disorders of the nervous system are associated with abnormal neural discharge patterns. One treatment regimen for such diseases and disorders includes drug therapy. Another treatment technique includes implanting in a patient's body a medical device having a pulse generator for electrically stimulating a target location of the patient's neural tissue. In one such available treatment for epilepsy, an electrical signal is applied to the vagus nerve by a neurostimulator device substantially as described in one or more of U.S. Pat. Nos. 4,702,254, 4,867,164, and 5,025,807, all of which are incorporated herein by reference.

Most embodiments of implantable medical devices incorporate the use or implantation of leads or wires to provide an electrical signal to electrodes coupled to a target tissue. To prevent the leads from moving freely once implanted in the patient-thereby increasing the risk of a stress-induced failure—the leads should be secured in some fashion. However, overly constraining the movement of the lead may increase transient stress and/or torsion in the lead, which, in turn, may cause fatigue wear to the lead.

Consequently, there is a need for an apparatus and methods for securing a lead extending from an implanted medical device within a patient while allowing the lead some freedom of motion, thereby reducing wear.

### SUMMARY OF THE DISCLOSED EMBODIMENTS

A lead tie-down for limiting movement of an electrical lead from an implantable medical device is disclosed. Some embodiments of the lead tie-down include a clamping member having a bore therethrough. The bore is configured to receive an electrical lead extending from an implantable medical device and to permit rotation of the lead within the clamping member.

Some system embodiments include a lead assembly having an electrical lead and a stimulating electrode and a lead tie-down coupled to the electrical lead and configured for coupling to surrounding tissue. The lead tie-down permits rotational movement of the electrical lead relative to the lead tie-down.

Some method embodiments include implanting a medical device into a patient, coupling an electrical lead extending from the medical device to the patient, and coupling a lead tie-down to the electrical lead and to surrounding tissue. The lead tie-down permits the electrical lead to rotate relative to the lead tie-down.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 schematically depicts an implantable medical device (IMD) system comprising a representative embodiment of a lead tie-down;

FIG. 2 schematically depicts the lead tie-down of FIG. 1 coupled to a lead having electrodes coupled to a target tissue;

FIG. 3 is a perspective view of the lead tie-down of FIG. 2; and

FIGS. 4A through 4D are various views of an embodiment of the clamping member of the lead tie-down of FIG. 3.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct f connection. Thus, if a first device couples to a second device, that connection may be through a direct connection, or through an indirect connection via other devices and connections.

### DETAILED DESCRIPTION OF THE PREFERRED EMBODIMENTS

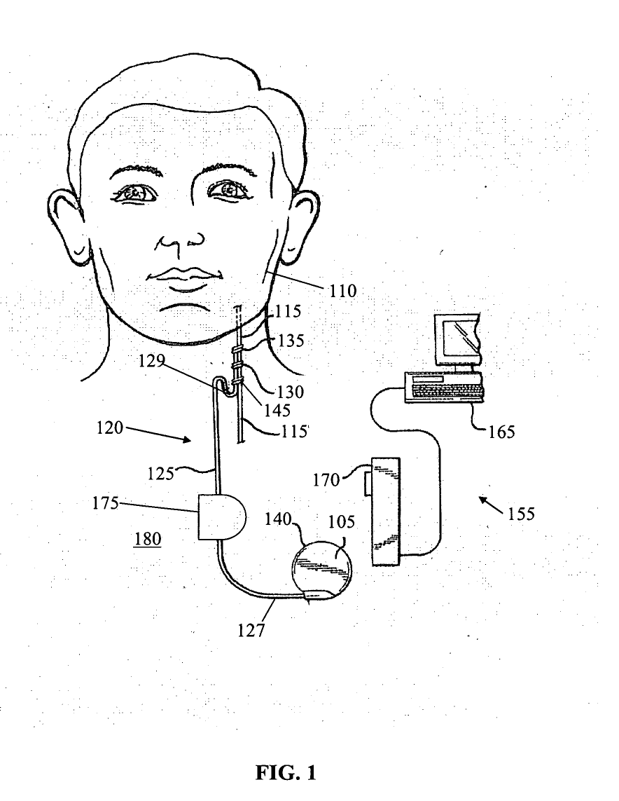

FIG. 1 schematically depicts an implantable medical device (IMD) system comprising an IMD 105 implanted within a patient 1 10, an embodiment of a lead tie-down 175 to secure a lead 125 extending from the IMD 105, and an external programming system 155 for programming the IMD 105. As shown, IMD 105 has been implanted in patient 110. IMD 105 is a representative implantable medical device and is shown coupled to a vagus nerve 115 of the patient 110. However, it will be understood that IMD 105 may be any of a variety of implantable medical devices and is not limited to a vagus nerve stimulation device. In at least one embodiment, including those depicted in FIG. 1, IMD 105 comprises a neurostimulator for applying an electrical signal to a neural structure in patient 110, specifically to a cranial nerve such as vagus nerve 115. As used herein “stimulate” and “modulate” both refer to the delivery of such an electrical signal to a target body structure, regardless of whether the signal causes a particular effect, for example, an induction of an action potential in a stimulated nerve, contraction of a muscle, etc.

Referring still to FIG. 1, lead assembly 120 is configured to deliver electrical signals from IMD 105 to vagus nerve 115. Lead assembly 120 comprises one or more leads 125, one or more electrodes 130, 135 and strain relief tether 145. Each lead 125 has a proximal end 127 that couples to IMD 105 and a distal end 129 on which electrodes 130, 135 are provided. Electrodes 130, 135 are coupled to vagus nerve 115. The outer housing (or “can”) 140 of IMD 105 is preferably electrically conductive, and thus may also function as an electrode. Electrodes 130, 135 may be used to stimulate the target tissue, which in this illustrative embodiment, is vagus nerve 115. Strain relief tether 145 comprises an attachment mechanism that couples lead assembly 120 to vagus nerve 115 in a manner that limits movement of distal end 127 of lead 125 relative to vagus nerve 115, thereby providing strain relief to lead assembly 120 at this location. An example of a suitable strain relief tether is described in U.S. Pat. No. 4,979,511, incorporated herein by reference.

External programming system 155 may be used to program the electrical signal generated by IMD 105 and applied to the target tissue, e.g., vagus nerve 115. External programming system 155 comprises programming device 165. In the embodiment shown in FIG. 1, programming device 165 is coupled to programming wand 170, which communicates with IMD 105 when placed on the patient's skin overlying the IMD. In systems where the external programming system 155 uses one or more channels in the Medical Implant Communications Service (MICS) bandwidths, the programming wand 170 may be omitted to permit more convenient communication directly between the programming device 165 and the IMD 105 and the pulse generator 110. Programming device 165 may comprise a personal computer, personal digital assistant (PDA) device, or other suitable computing devices consistent with the description contained herein. IMD 105 and wand 170 both include a transceiver. The transceivers in IMD 105 and wand 170 permit signals to be communicated wirelessly and non-invasively between these components. Thus, programming system 155 is capable of transmitting one or more parameters to IMD 105 to define the electrical signal to be applied to the target tissue.

Given the function of IMD 105, it is therefore desirable to protect IMD 105, and its related components, from unnecessary wear. To that end, lead tie-down 175 is coupled to lead 125 and to surrounding tissue 180. Lead tie-down 175 permits limited movement of lead 125 relative to surrounding tissue 180. Permitting rotational movement of lead 125 relative to tie-down 175, in contrast to conventional tie-down methods that do not permit rotational movement of the leads, reduces transient stresses—and thus wear—to lead 125. In turn, reduced wear to lead 125 increases the service life of the lead by reducing the likelihood of a lead break. Moreover, permitting rotational movement of lead 125 relative to tie-down 175, reduces transient stresses and thus potentially reduces stresses/loads transmitted to the vagus nerve 115. In turn, reduced stresses/loads transmitted to the vagus nerve 115 reduces the likelihood of adverse medical events related to loading the neural structure.

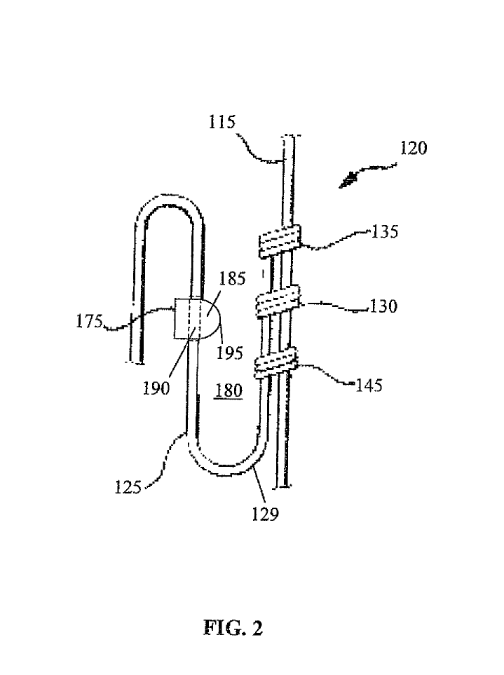

FIG. 2 schematically depicts lead tie-down 175 coupled to lead 125 of IMD 105 and surrounding tissue 180. As described above, lead 125 comprises distal end 129 on which electrodes 130, 135 are provided. Each of electrodes 130, 135 is coupled to the target tissue, (e.g., vagus nerve 115) and provides electrical stimulation to the target tissue from IMD 105. Lead tie-down 175 comprises a clamping member 185 surrounding a bearing element 190. When implanted to secure lead 125, bearing element 190 of lead tie-down 175 couples to lead 125 in a manner to be described below, while end 195 of clamping member 185 is sutured to surrounding tissue 180. In some embodiments, clamping member 185 may comprise rubber or other similar material that permits sutures to be inserted directly through clamping member 185. In other embodiments, clamping member 185 may further comprise small holes or apertures through which sutures are inserted to secure clamping member 185 to the surrounding tissue.

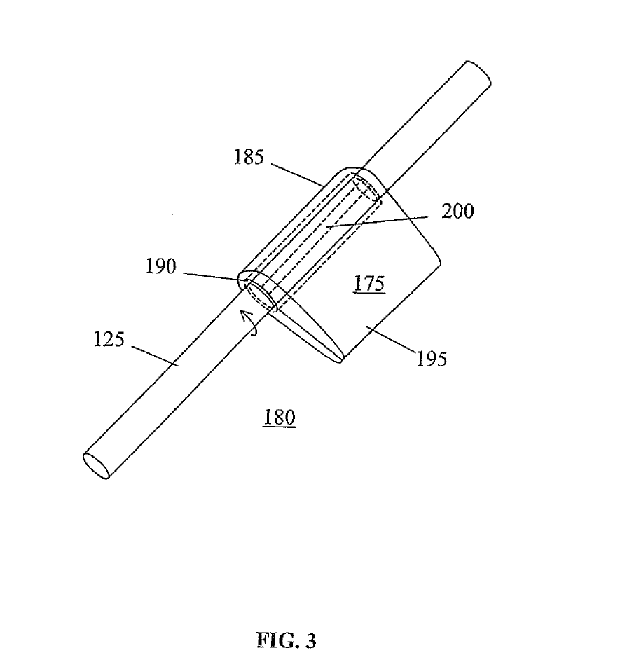

Turning now to FIG. 3, in some embodiments, bearing element 190 of lead tie-down 175 comprises a sleeve with a flexible slot 200 extending along the length of the sleeve. Bearing element 190 preferably comprises a biocompatible material(s), such as titanium, stainless steel, or biocompatible polymers such as silicone, PTFE (teflon), etc. Slot 200 may be formed by machining, injection molding, or other suitable method known in the industry. Lead 125 may be inserted through flexible slot 200 into the sleeve, or the bearing element 190, and bearing element 190 then inserted into clamping member 185.

Bearing element 190 is shaped to enable lead 125 to freely rotate within the bore of clamping member 185. To further enable such rotation, the outer surface of bearing element 190, the bore of clamping member 185, or both may be coated with Teflon, or other similar material. Moreover, the inner surface of bearing element 190 preferably comprises a material or coating suitable for engaging lead 125 such that, once the lead tie-down 175 is implanted, bearing element 190 and lead 125 have limited translational movement relative to each other.

Although the illustrated embodiment depicts bearing element 190 as cylindrically shaped, in other embodiments bearing element 190 may assume different shapes, all of which enable lead 125 to freely rotate within clamping member 185. Also, in some embodiments, the sleeve, or bearing element 190, further comprises end caps that after bearing element 190 is installed within clamping member 185, prevent bearing element 190 from translating relative to and disengaging clamping member 185. In other embodiments, the outer surface of bearing element 190 may be configured to limit translational movement of bearing element 190 relative to clamping member 185. For example, cilia or other similar components may be attached along the outer surface of bearing element 190. Such components will enable rotational movement, while at the same time limiting translational movement, of bearing element 190 within clamping member 185.

In other embodiments of the lead tie-down 175, the surface that defines the bore of clamping member 185 is the bearing element 190. In these embodiments, a slot 200 is not needed. Instead, lead 125 may be inserted into the bore of clamping member 185. The bore of clamping member 185 is shaped to enable lead 125 to freely rotate within the clamping member 185. To further enable such rotation, the lead 125, the bore of clamping member 185, or both may be coated with Teflon, or other similar material.

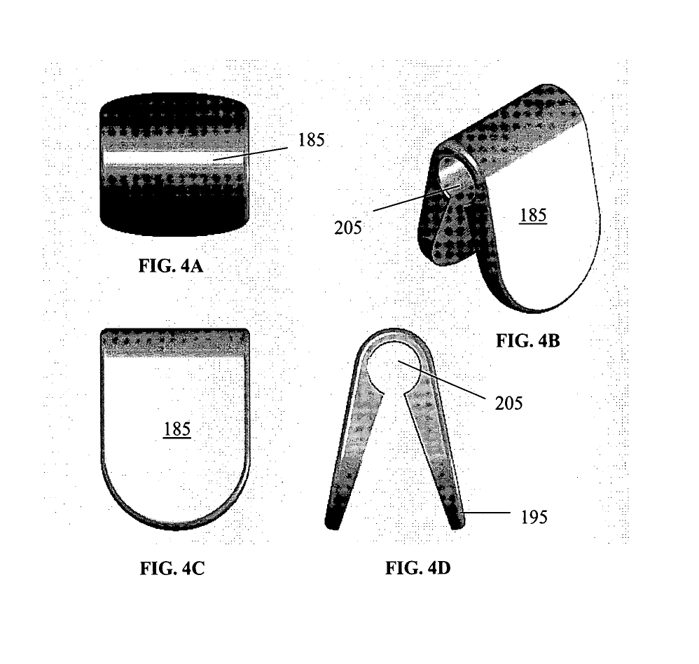

Referring now to FIGS. 4A through 4D, an embodiment of clamping member 185 is depicted, comprising a clamshell shaped housing having a bore 205 therethrough to accommodate bearing element 190. Although the illustrated embodiments depict clamping member 185 as clamshell-shaped, in other embodiments clamping member 185 may assume different shapes, all comprising a bore 205 therethrough to accommodate bearing element 190. Like bearing element 190, clamping member 185 also comprises biocompatible material(s), such as silicone polymers, Teflon, etc.

As described above, in some embodiments, bearing element 190 is the inner surface of clamping member 185 defining bore 205. In other embodiments, bearing element 190 is a sleeve that may be inserted into bore 205 of clamping member 185. In embodiments of the lead tie-down 175 wherein bearing element 190 is a sleeve, clamping member 185 may further comprise a lip on each end of bore 205 to prevent bearing element 190 from translating relative to, and disengaging from, clamping member 185 once bearing element 190 is installed within bore 205 of clamping member 185.

During implantation, lead tie-down 175 is first coupled to lead 125 of IMD 105. Lead 125 is inserted through slot 200 (FIG. 3) of bearing element 190, permitting bearing element 190 to be positioned concentrically about lead 125. In the embodiment of FIG. 4, clamping member 185 is opened, to enable access to bore 205. Bearing element 190, which is concentric about lead 125, is then positioned concentrically inside bore 205 of clamping member 185. Clamping member 185 is then closed about bearing element 190, to complete coupling of lead tie-down 175 to lead 125. Next, end 195 of clamping member 185 is sutured to surrounding tissue 180 to secure lead tie-down 175 in position. Once closed, the hinge stiffness of clamping member 185, and the suture member threaded through end 195, prevent clamping member 185 from inadvertently opening. In some embodiments, clamping member 185 may include an interlocking structure such that once closed, clamping member 185 will not inadvertently open.

Once implanted, lead tie-down 175 preferably permits only limited movement of lead 125. Specifically, in one embodiment lead tie-down 175 prevents lead 125 from translating within bearing element 190. Thus, translational movement of lead 125 between the implanted location of lead tie-down 175 and vagus nerve 115 is limited. As a result, transient stresses in lead 125 resulting from continual shifting of lead 125 are reduced, particularly at and near the couplings of electrodes 130,135 to vagus nerve 115. This reduces the likelihood that the couplings of lead 125 to vagus nerve 115 will fatigue over time and break.

At the same time, lead 125 is permitted to rotate freely within bearing element 190 of lead tie-down 175. The benefit to this becomes clear by considering what happens when a lead extending from an IMD is fully constrained, meaning the lead is constrained in a way that allows neither rotational nor translational movement of the lead at the point where the lead is constrained. As a result, any movement of the lead about the point where it is constrained creates high stress levels in the lead at that location. Over time, these transient stress levels fatigue the lead at this location and may eventually cause the lead to break. Thus, by allowing lead 125 to rotate within bearing element 190 of lead tie-down 175, such high stress locations are reduced or effectively eliminated.

Therefore, by permitting limited movement of lead 125, lead tie-down 175 permits reduced stress levels to lead 125, relative to stress levels resulting from conventional methods or devices used to secure a lead extending from an IMD. Reducing transient stresses allows for less fatigue wear to, and thus, a longer service life for, lead 125.

While embodiments of the invention have been shown and described, modifications thereof can be made by one skilled in the art without departing from the spirit and teachings of the invention. The embodiments described and the examples provided herein are exemplary only, and are not intended to be limiting. For example, the embodiments described and illustrated herein depict a lead tie-down for securing an electrical lead extending from an IMD used for vagus nerve stimulation (VNS) therapy. In other embodiments, the lead tie-down may be used to secure an electrical lead from another type of IND. Furthermore, in still other embodiments, the lead tie-down may be used to secure another type of lead. Thus, many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of any reference in the Description of the Related Art is not an admission that such reference is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. A medical device system, comprising:
a lead assembly having an electrical lead and a stimulating electrode; and
a lead tie-down coupled to the electrical lead and configured for coupling to surrounding tissue;
wherein said lead tie-down permits rotational movement of the electrical lead relative to said lead tie-down.

2. The system of claim 1, wherein said lead tie-down is configured for suturing to the surrounding tissue.

3. The system of claim 1, wherein the lead tie-down further comprises:
a clamping member having a bore therethrough; and
a bearing element;
wherein said bearing is configured to receive the electrical lead and to permit rotation of the lead within the bore relative to said clamping member.

4. The system of claim 3, wherein said bearing element limits translational movement of the lead relative to the lead tie-down.

5. The system of claim 3, wherein at least one of said bearing element and the electrical lead is coated with a material to further enable rotation of the lead relative to the lead tie-down.

6. The system of claim 5, wherein the coating comprises polytetrafluoroethylene.

7. The system of claim 3, wherein said bearing element is a surface of the clamping member that defines the bore.

8. The system of claim 3, wherein said clamping member further comprises a hinged housing configured to open to receive said lead within the bore and to close to contain said lead within the bore.

9. A lead tie-down apparatus for an implantable medical device, comprising:
a clamping member having a bore therethrough, the bore configured to receive an electrical lead extending from an implantable medical device and to permit rotation of the lead within said clamping member.

10. The apparatus of claim 9, wherein the clamping member comprises a biocompatible material.

11. The apparatus of claim 9, further comprising a bearing element having a surface in contact with the electrical lead, said bearing element configured to receive the electrical lead and comprises material having a friction coefficient that causes the electrical lead and said bearing element to rotate together as a single unit within the bore of said clamping member.

12. The apparatus of claim 11, wherein said bearing element is a surface of the clamping member that defines the bore.

13. The apparatus of claim 11, wherein said bearing element is a cylindrical sleeve inserted into the bore of said clamping member.

14. The apparatus of claim 13, wherein said bearing element comprises a slot formed along its length to enable said electrical lead to be inserted within said bearing element.

15. The apparatus of claim 13, wherein at least one of a surface of said bearing element in contact with said clamping member and a surface of said clamping member in contact with said bearing element comprises polytetrafluoroethylene.

16. The apparatus of claim 9, wherein said clamping member further comprises a hinged housing configured to open to receive said electrical lead within the bore and to close to contain said lead within the bore.

17. A method, comprising:
implanting a medical device into the body of a patient;
coupling an electrical lead extending from the medical device to a target tissue in the patient's body; and
coupling a lead tie-down to the electrical lead and to surrounding tissue;
wherein the lead tie-down permits the electrical lead to rotate relative to the lead tie-down.

18. The method of claim 17, wherein said lead tie-down limits translational movement of the electrical lead relative to said tie-down.

19. The method of claim 17, wherein the coupling of the lead tie-down to the electrical lead comprises:
opening a hinged housing having a bore therethrough;
aligning the electrical lead within the bore of the hinged housing; and
closing the hinged housing to contain the electrical lead within the bore.

20. The method of claim 19, further comprising the steps of inserting a sleeve into said bore and inserting said electrical lead into said sleeve.
